(19) United States
(12) Patent Application Publication
(10) Pub. No.: US 2023/0212683 A1
(43) Pub. Date: Jul. 6, 2023

(54) MICROSATELLITE INSTABILITY SIGNATURES

(71) Applicant: Personal Genome Diagnostics Inc., Baltimore, MD (US)

(72) Inventor: Derrick Wood, Baltimore, MD (US)

(21) Appl. No.: 17/909,537

(22) PCT Filed: Mar. 10, 2021
(86) PCT No.: PCT/US2021/021769

§ 371 Date: Sep. 6, 2022

Related U.S. Application Data
(60) Provisional application No. 62/988,571, filed on Mar. 12, 2020.

(51) Int. Cl. C12Q 1/6886; A61K 39/395

(52) U.S. Cl. CPC C12Q 1/6886; A61K 39/395; C12Q 2600/112; C12Q 2600/156

(57) ABSTRACT

Provided herein are methods for determining microsatellite instability (MSI) based on tracts of nucleotide repeats and somatic mutations in nucleic acid from patient samples. The methods provided herein include determining genomic signature scores and MSI scores for classification of samples as MSI or microsatellite-stable (MSS). The methods provided herein are useful for selecting patients for immunotherapy.

### CROSS REFERENCE TO RELATED APPLICATIONS

This application claims the benefit under 35 USC § 119(e) to U.S. Application Serial No. 62/988,571 filed Mar. 12, 2020, now pending. The disclosure of the prior application is considered part of and is incorporated by reference in the disclosure of this application.

### FIELD OF THE INVENTION

The invention relates generally to the detection of microsatellite instability (MSI) and more specifically to analysis of nucleotide tract repeat lengths and genomic signatures of somatic mutations.

### INCORPORATION OF SEQUENCE LISTING

The material in the accompanying sequence listing is hereby incorporated by reference into this application. The accompanying sequence listing text file, name PGDX3100-1WO_SL.txt, was created on Mar. 8, 2021, and is 750 bytes. The file can be accessed using Microsoft Word on a computer that uses Windows OS.

### BACKGROUND INFORMATION

Microsatellite instability (MSI) in tumors is a biomarker that guides the selection of immunotherapies, such as checkpoint inhibitor therapy, for treatment of patients. Microsatellite instability can be detected by analysis of next-generation sequencing (NGS) data and/or analysis of amplified fragment lengths. These assays examine sequenced or amplified microsatellite length distributions. However, microsatellites are known to be difficult to amplify or sequence with high accuracy.

Individual substitutions can be detected with accuracy at lower limits of detection than individual microsatellite lengths. For example, somatic mutations are often located in regions of the genome that are easier to accurately sequence than microsatellites.

### SUMMARY OF THE INVENTION

The present invention relates to detection of microsatellite instability using analysis of microsatellite tracts and somatic mutations.

Provided herein, in some embodiments, are methods of determining microsatellite instability (MSI) including: (i) determining the presence of somatic allele lengths in a plurality of tracts of nucleotide repeats in sequenced DNA in a sample obtained from a subject; (ii) determining the presence of somatic mutations in the DNA in regions outside of the plurality of tracts of nucleotide repeats; (iii) determining a fit of genomic signatures of the somatic mutations found outside of the plurality of tracts of nucleotide repeats to genomic signatures of mismatch repair deficiency; (iv) applying a rule to the results of the determining steps to obtain an MSI score; and (v) classifying the sample as microsatellite instability-high (MSI-H) or microsatellite-stable (MSS) based on the MSI score.

In some embodiments, the methods provided herein further include preparing a report including the MSI score and a classification of the sample as microsatellite instability-high (MSI-H) or microsatellite-stable (MSS). In some embodiments, the sample is a tumor sample.

In some embodiments, the plurality of tracts of nucleotide repeats of the methods provided herein include mononucleotide repeats, dinucleotide repeats, trinucleotide repeats, tetranucleotide repeats, pentanucleotide repeats, hexanucleotide repeats, heptanucleotide repeats, octanucleotide repeats, or combinations thereof. In some embodiments, the plurality of tracts of nucleotide repeats include mononucleotide repeats. In some embodiments, tracts of nucleotide repeats in MSI-H samples are shorter relative to tracts of nucleotide repeats in reference samples. In some embodiments, tracts of nucleotide repeats in MSI-H samples are at least two base pairs shorter relative to tracts of nucleotide repeats in reference samples. In some embodiments, the reference samples are matched normal samples or MSS samples.

In some embodiments, the methods provided herein further include determining a frequency of shorter tracts of nucleotide repeats. In some embodiments, the plurality of tracts of nucleotide repeats of the methods provided herein includes one or more microsatellite markers. In some embodiments, the one or more microsatellite markers are selected from human genome microsatellite markers. In some embodiments, the one or more microsatellite markers are selected from the group consisting of: BAT-25; BAT-26; MONO-27; NR-21; NR-24; and any combination thereof. In some embodiments, the one or more microsatellite markers are selected from the group consisting of: BAT-25; BAT-26; D5S346; D2S123; D17S250; and any combination thereof. In some embodiments, the one or more microsatellite markers are selected from the group consisting of BAT-25; BAT-26; MONO-27; NR-21; NR-24; D5S346; D2S123; D17S250; BAT40; and any combination thereof.

In some embodiments, the genomic signatures of the somatic mutations of the methods provided herein include eleven base pairs surrounding the somatic mutations. In some embodiments, the methods provided herein further include assigning a genomic signature score to the somatic mutations. In some embodiments, somatic mutations are classified as associated with an MSI-H sample or an MSS sample based on the genomic signature score.

In some embodiments, the methods provided herein further include selecting or administering a treatment to the patient based on the MSI score. In some embodiments, the MSI score indicates that the sample of the patient is microsatellite instability-high (MSI-H) and the treatment comprises an immune checkpoint inhibitor. In some embodiments, the immune checkpoint inhibitor includes an antibody. In some embodiments, the antibody is selected from the group consisting of: an anti-PD-1 antibody; an anti-IDO antibody; an anti-CTLA-4 antibody; an anti-PD-L1 antibody; and an anti-LAG-3 antibody. In some embodiments, the checkpoint inhibitor is Pembrolizumab (KEYTRUDA®), Nivolumab (OPDIVO®), Atezolizumab (TECENTRIQ®), or Ipilimumab (YERVOY®).

In some embodiments, sequenced DNA includes one or more sequenced genomes, one or more sequenced exomes, or regions of one or more sequenced genomes or one or more sequenced exomes.

### BRIEF DESCRIPTION OF THE DRAWINGS

FIG. 1 illustrates data analysis at more than one dimension.

FIG. 2 illustrates that high-dimensionality data requires a multitude of training data.

FIG. 3 illustrates PGDx Cerebro, a machine learning approach for somatic mutation discovery.

FIG. 4 illustrates detection of short alleles at mononucleotide tracts.

FIG. 5 illustrates detection of mismatch repair deficiency mutation signatures (Catalogue of Somatic Mutations in Cancer (COSMIC), available at https://cancer.sanger.ac.uk/cosmic/signatures_v2; Wellcome Sanger Institute).

FIG. 6 illustrates the concordance between exome signature scores and PCR. Representative exome data is shown.

FIG. 7 illustrates results obtained combining detection of short alleles at mononucleotide tracts and detection of mismatch repair deficiency mutation signatures.

FIG. 8 illustrates linear separability of data obtained using an ensemble method of MSI detection.

### DETAILED DESCRIPTION OF THE INVENTION

Many cancers involve the accumulation of mutations that are the result of mismatch repair (MMR) deficiency. An important marker of MMR deficiency is microsatellite instability (MSI). The presence of MMR deficiency or MSI may serve as a marker for responsiveness to immunotherapy such as checkpoint inhibitor therapy, for example. The methods provided herein are useful for the detection of MSI and selection of therapeutic regimens.

In some embodiments, the methods provided herein include determining microsatellite instability (MSI) including: (i) determining the presence of somatic allele lengths in a plurality of tracts of nucleotide repeats in sequenced DNA in a sample obtained from a subject; (ii) determining the presence of somatic mutations in the DNA in regions outside of the plurality of tracts of nucleotide repeats; (iii) determining a fit of genomic signatures of the somatic mutations found outside of the plurality of tracts of nucleotide repeats to genomic signatures of mismatch repair deficiency; (iv) applying a rule to the results of the determining steps to obtain an MSI score; and (v) classifying the sample as microsatellite instability-high (MSI-H) or microsatellite-stable (MSS) based on the MSI score.

The methods provided herein can include ensemble methods. As used herein, “ensemble method” refers to use of results or data from more than one classification system to arrive at a final classification. Multiple classification systems can be used in the methods provided herein to arrive at a final classification. Any number of classification systems can be used to arrive at a final classification, such as 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, and any number or range in between, or more classification systems. In some embodiments, the methods provided herein use data from two classification systems. In some embodiments, the two classification systems are tract-based and mutation signature-based classification systems.

As used herein, the term “MSI-H” means “microsatellite instability-high.” As used herein, the term “MSI” means “microsatellite-instable” or “microsatellite instability,” as indicated by context. As used herein, the term “MSI-L” means “microsatellite instability-low.” As used herein, the term “MSS” means “microsatellite-stable.” MSI status can be used to classify tumors and/or tumor samples into groups of tumors that include microsatellite unstable and microsatellite stable tumors. As used herein, an MSI tumor is a tumor or sample with a higher degree of microsatellite instability relative to normal or non-tumor tissue or relative to an MSI-L or MSS tumor or sample. Accordingly, an MSI tumor sample is a sample with a higher degree of microsatellite instability relative to a normal or non-tumor sample or a sample from an MSI-L or MSS tumor. As used herein, the terms “MSI” and “MSI-H” can be used interchangeably when referring to MSI status of a tumor or a tumor sample, unless context clearly indicates otherwise.

Any number of nucleotide repeat tracts can be analyzed using the methods provided herein. Exemplary numbers of nucleotide repeat tracts that can be analyzed include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 150, at least 200, at least 250, at least 300, at least 350, at least 400, at least 450, at least 500, at least 600, at least 700, at least 800, at least 900, at least 1000, and any number or range in between, or more nucleotide repeat tracts.

### Sequencing

In some embodiments, the methods provided herein include sequenced DNA in a sample obtained from a patient. Any sequencing method can be used, including Sanger sequencing using labeled terminators or primers and gel separation in slab or capillary systems, and Next Generation Sequencing (NGS), for example. Exemplary NGS methodologies include the Roche 454 sequencer, Life Technologies SOLiD® systems, the Life Technologies Ion Torrent, and Illumina systems such as the Illumina Genome Analyzer II, Illumina MiSeq, Illumina HiSeq, Illumina NextSeq, and Illumina NovaSeq instruments.

In some embodiments, sequenced DNA in the sample comprises one or more sequenced genomes, or regions thereof. In some embodiments, sequenced DNA in the sample comprises one or more sequenced exomes, or regions thereof. As used herein, the term “exome sequencing” refers to sequencing all protein coding exons of genes in a genome. Exome sequencing can include target enrichment methods such as array-based capture and in-solution capture of nucleic acid, for example.

### Nucleotide Repeats

In some embodiments, the methods provided herein include determining the presence of somatic allele lengths in a plurality of tracts of nucleotide repeats in the DNA from a patient sample. Nucleotide repeats can constitute a microsatellite or short tandem repeat (STR). Accordingly, as used herein, the terms “microsatellite” and “microsatellite marker” refer to polymorphic DNA loci that contain repeating nucleotide sequences. Nucleotide repeats can be of any length. Any microsatellite marker or combination of microsatellite markers can be analyzed by the methods provided herein. In some embodiments, the microsatellite markers are present in the human genome, although a person of skill in the art will appreciate that microsatellite markers of any species can be analyzed by the methods provided herein. Microsatellite markers for analysis can be identified by scanning a reference genome for nucleotide repeats, for example.

Typically, a DNA motif of a nucleotide repeat is repeated five to 50 times, for example, although fewer or more repetitions are possible. Further, a tract of nucleotide repeats can have any number of nucleotides that are repeated. For example, nucleotide repeats can include one to six base pairs or up to ten base pairs as a repeating DNA motif. In some embodiments, the plurality of tracts of nucleotide repeats comprises mononucleotide repeats, dinucleotide repeats, trinucleotide repeats, tetranucleotide repeats, pentanucleotide repeats, hexanucleotide repeats, heptanucleotide repeats, octanucleotide repeats, or combinations thereof. In some embodiments, the plurality of tracts of nucleotide repeats comprises mononucleotide repeats. Microsatellite markers for analysis can be identified by scanning a reference genome for mononucleotide repeats and/or repeats of any other length, for example.

A nucleotide repeat in DNA from a patient’s sample can differ in length from a nucleotide repeat in a reference sample by any number of nucleotides. Differences in nucleotide repeat length can result from different numbers of a repeated DNA motif. As an example, nucleotide repeat lengths in an MSI or MSI-H sample or tumor can be shorter than nucleotide repeat lengths in a reference sample or tumor. As another example, nucleotide repeat lengths in an MSI or MSI-H sample or tumor can be longer than nucleotide repeat lengths in a reference sample or tumor. As yet another example, partial repeats of a repeated DNA motif, deletions within a repeated DNA motif, insertions within a repeated DNA motif, substitutions within a repeated DNA motif, or any combination thereof, can alter the length of a nucleotide repeat. Nucleotide repeat lengths can be longer or shorter in an MSI or MSI-H sample or tumor relative to a reference sample or tumor by any number of nucleotides or nucleotide repeats. In some embodiments, tracts of nucleotide repeats in MSI or MSI-H samples are shorter relative to tracts of nucleotide repeats in one or more reference samples. In some embodiments, tracts of nucleotide repeats in MSI or MSI-H samples are at least two base pairs shorter relative to tracts of nucleotide repeats in one or more reference samples. In some embodiments, the one or more reference samples are matched normal samples or MSS samples.

In some embodiments, the methods provided herein further include determining a frequency of shorter tracts of nucleotide repeats. For example, the frequency of shorter tracts of nucleotide repeats can be greater for an MSI or MSI-H sample or tumor relative to the frequency of shorter tracts of nucleotide repeats in one or more reference samples. As an example, any altered allele, such as shorter or longer tracts of nucleotide repeats, can occur 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 550%, 600%, 650%, 700%, 750%, 800%, 850%, 900%, 950%, 1000%, and any number or range in between, more frequently relative to a reference allele.

The proportion of analyzed nucleotide repeat tracts that are unstable can be used to classify a sample as an MSI or MSI-H sample. As used herein, the term “unstable” when referring to nucleotide repeat tracts means that the length of nucleotide repeat tracts in a sample can vary. For example, nucleotide repeat tracts can be shorter or longer. In some embodiments, the length of nucleotide repeat tracts in a sample varies as compared to the length of nucleotide repeat tracts in a reference sample. In some embodiments, the length of nucleotide repeat tracts varies as compared to the length of germline alleles of the nucleotide repeat tracts. In some embodiments, the length of nucleotide repeat tracts in a sample is shorter as compared to the length of nucleotide repeat tracts in a reference sample. In some embodiments, the length of nucleotide repeat tracts in a sample is shorter as compared to the length of germline alleles of the nucleotide repeat tracts. In some embodiments, the length of nucleotide repeat tracts is longer as compared to the length of nucleotide repeat tracts in a reference sample. In some embodiments, the length of nucleotide repeat tracts in a sample is longer as compared to the length of germline alleles of the nucleotide repeat tracts.

The presence of about 2, about 3, about 4, about 5, about 6, about 7, about 8, about 9, about 10, about 11, about 12, about 13, about 14, about 15, or more shorter nucleotide repeat tracts of about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 or more nucleotide repeat tracts analyzed can be used to classify a sample as an MSI or MSI-H sample. In some embodiments, the presence of about 8 nucleotide repeat tracts having shorter alleles out of about 68 nucleotide repeat tracts analyzed is used to classify a sample as an MSI or MSI-H sample. In some embodiments, the presence of about 11 nucleotide repeat tracts having shorter alleles out of about 68 nucleotide repeat tracts analyzed is used to classify a sample as an MSI or MSI-H sample. In some embodiments, a sample is classified as an MSI or MSI-H sample when all nucleotide repeat tracts analyzed have shorter alleles. In some embodiments, the sample is a tumor sample.

In some embodiments, the plurality of tracts of nucleotide repeats comprises one or more microsatellite markers. Exemplary microsatellite markers include BAT-25, BAT-26, MONO-27, NR-21, NR-24, BAT-40, TGFβ RII, IGFIIR, hMSH3, BAX and dinucleotide loci such as D2S123, D9S283, D9S1851, D2S123, D17S250, and D18S58. In some embodiments, the one or more microsatellite markers are selected from the group consisting of: BAT-25; BAT-26; MONO-27; NR-21; NR-24; and any combination thereof. In some embodiments, the one or more microsatellite markers are selected from the group consisting of: BAT-25; BAT-26; D5S346; D2S123; D17S250; and any combination thereof. In some embodiments, the one or more microsatellite markers are selected from the group consisting of BAT-25; BAT-26; MONO-27; NR-21; NR-24; D5S346; D2S123; D17S250; BAT40; and any combination thereof.

### Detection of Somatic Mutations

In some embodiments, the methods provided herein include determining the presence of somatic mutations in DNA from a patient sample. The presence of somatic mutations can be determined in DNA in regions outside of a plurality of tracts of nucleotide repeats.

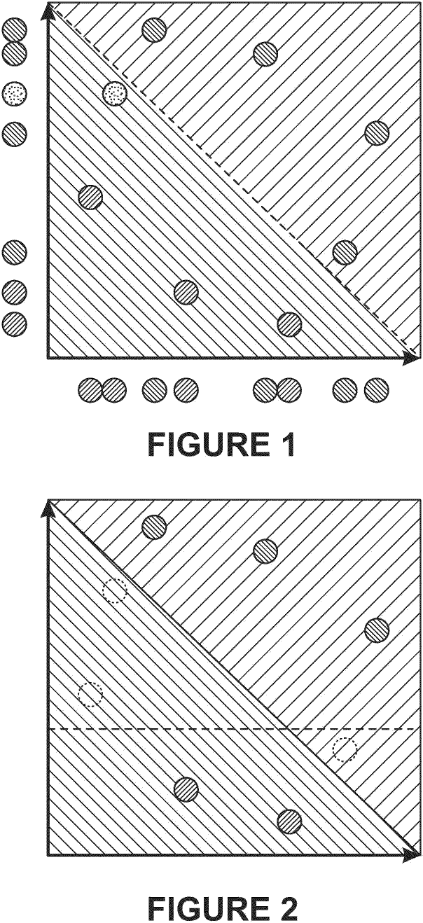

Looking at a single dimension of data may not be sufficient for separation of the data into distinct classes. Rather, it is often necessary to examine multiple dimensions of data to allow for separation of the data into correct classes (FIG. 1).

Somatic mutation detection may require many dimensions because of artifacts, such as lab artifacts, sequencing artifacts, and alignment artifacts that potentially confound analysis. Lab artifacts can be detected by strand bias, genomic context, and combination of reference and alternative alleles, for example. Sequence artifacts can be detected by poor base quality or by genomic context, for example. Exemplary parameters that can contribute to detection of alignment artifacts can include mapping quality, aligner agreement, and positional distribution, for example. A further confounding aspect in detecting somatic mutations is the possible presence of germline mutations. Germline mutations may be present in an individual not identified as having a mutation, i.e., normals, for example. In addition, germline mutations may be found in databases, for example.

High dimensionality data can require a multitude of training data. For example, without sufficient and representative training data, a classifier may fit improperly, leading to overfitting (FIG. 2). Machine learning methods can much more easily and accurately fit large datasets than humans. A machine model designated Cerebro has been developed that examines over 70 dimensions. Moreover, for exome data, over 2 million labeled candidate mutations are used with this model.

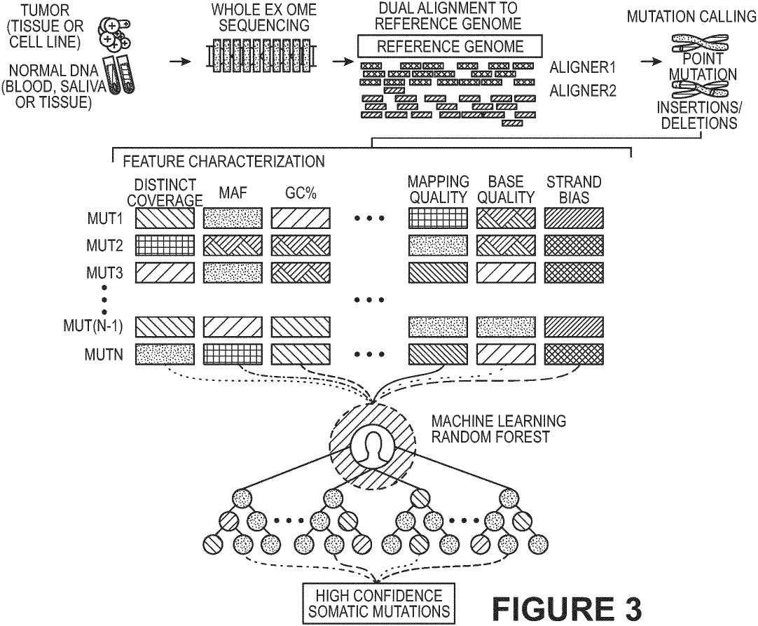

The features of Cerebro for use in somatic mutation detection are illustrated in FIG. 3 (Wood et al., Sci. Transl. Med. 2018, Vol. 10, eaar7939). Cerebro uses a machine learning model to score candidate mutations and separate true somatic mutations from germline mutations and artifacts. For example, dual alignment increases data for statistical analysis per mutation by providing data from two alignments. Additional characteristics considered by Cerebro include analysis of mutation context, such as %GC and DUST (a measurement of sequence complexity), for example, sample coverage, such as distinct coverage and mutant allele frequency (MAF), sequence and/or alignment quality, such as mapping quality and base quality, for example, and coverage comparison, including analysis of tumor and normal samples and application of statistics such as Fisher’s exact test (FET), for example. Accordingly, in some embodiments, detecting somatic mutations in the methods provided herein includes using Cerebro.

### Genomic Signatures of Somatic Mutations

In some embodiments, the methods provided herein include determining genomic signatures of somatic mutations. In some embodiments, the methods provided herein include determining the fit of genomic signatures of somatic mutations found outside of a plurality of tracts of nucleotide repeats to genomic signatures of mismatch repair deficiency. A genomic signature can include any sequence context of the somatic mutation, including sequences adjacent to the somatic mutation, sequences surrounding the somatic mutation, or sequences that include the somatic mutation. Sequences adjacent to a somatic mutation can be located 5′ and/or 3′ of the somatic mutation and extend for any number of base pairs from the somatic mutation. As used herein, the term “adjacent” refers to sequences directly next to a somatic mutation. In some embodiments, sequences that make up sequence context or a genomic signature of a somatic mutation can be located at a distance from the somatic mutation. For example, sequences that make up sequence context or a genomic signature of a somatic mutation may not be located directly adjacent to the somatic mutation, but located at a distance of 1 base pair, 2 base pairs, 3 base pairs, 4 base pairs, 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, 20 base pairs, 25 base pairs, 30 base pairs, 35 base pairs, 40 base pairs, 45 base pairs, 50 base pairs, 55 base pairs, 60 base pairs, 65 base pairs, 70 base pairs, 75 base pairs, 80 base pairs, 85 base pairs, 90 base pairs, 95 base pairs, 100 base pairs, 125 base pairs, 150 base pairs, 200 base pairs, 250 base pairs, 300 base pairs, 350 base pairs, 400 base pairs, 450 base pairs, 500 base pairs, 600 base pairs, 700 base pairs, 800 base pairs, 900 base pairs, 1000 base pairs, 5000 base pairs, 10 kilobase pairs (kbp), 20 kbp, 30 kbp, 40 kbp, 50 kbp, 60 kbp, 70 kbp, 80 kbp, 90 kbp, 100 kbp, 200 kpb, 300 kbp, 400 kbp, 500 kbp, 600 kpb, 700 kbp, 800 kbp, 900 kbp, 1 megabase pair (Mbp), and any number or range in between, from the somatic mutation. Such sequences can be located on either side, i.e., 5′ or 3′, or on both sides, i.e., 5′ and 3′, of the somatic mutation. For sequences that are located either 5′ or 3′ or both 5′ and 3′ of the somatic mutation, the sequence context can include the somatic mutation. In some embodiments, for sequences that are located both 5′ and 3′ of the somatic mutation, the sequence context surrounds the somatic mutation.

Sequence context or a genomic signature can include any number of base pairs, such as 1 base pair, 2 base pairs, 3 base pairs, 4 base pairs, 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, 20 base pairs, 25 base pairs, 30 base pairs, 35 base pairs, 40 base pairs, 45 base pairs, 50 base pairs, 55 base pairs, 60 base pairs, 65 base pairs, 70 base pairs, 75 base pairs, 80 base pairs, 85 base pairs, 90 base pairs, 95 base pairs, 100 base pairs, 125 base pairs, 150 base pairs, 200 base pairs, 250 base pairs, 300 base pairs, 350 base pairs, 400 base pairs, 450 base pairs, 500 base pairs, 600 base pairs, 700 base pairs, 800 base pairs, 900 base pairs, 1000 base pairs, and any number or range in between. In some embodiments, genomic signatures of the somatic mutations include eleven base pairs surrounding the somatic mutations.

The methods provided herein can include assigning a genomic signature score to somatic mutations. In some embodiments, somatic mutations are classified as associated with an MSI-H or MSI sample or an MSS sample based on the genomic signature score. For example, a set of position weight matrices (PWMs) can be used that evaluates the log-likelihood that a particular single base substitution (SBS) is from an MSI or MSI-H tumor given the reference allele, alternate allele, and 11 nt context or genomic signature centered on the reference base (Example 2, below).

Genomic signatures of somatic mutations can include any structural context. Accordingly, methods provided herein can include determining nucleic acid structural context of somatic mutations. As used herein, “structural context” means primary structure, secondary structure, tertiary structure, quaternary structure, and any combination thereof. For example, primary structure can include sequence context. Secondary structure can include interaction between bases, such as formation of double-stranded regions, stem-loops, hairpin loops, tetraloops, and pseudoknots, for example. Tertiary structure can include large-scale folding and interaction of secondary structures, such as interaction of stem-loops, hairpin loops, and other secondary structural features, for example. Quaternary structure can include interaction between nucleic acid molecules and proteins, such as organization of DNA into nucleosomes, for example, and interaction between separate nucleic acids, for example.

As an example, primary structure context or sequence can affect the tendency of a base to be mutated by a particular mutational process, such as mismatch repair deficiency and others. As another example, secondary structure context can affect the tendency of a base to be mutated by a particular mutational process, such as mismatch repair deficiency and others. As yet another example, distance between an exonic base and a splice site, such as the nearest splice site, for example, can affect the tendency of the base to be mutated by a particular mutational process, such as mismatch repair deficiency and others. Structure context can also affect the relative location of somatic mutations. For example, somatic mutations can accumulate in the center or interior of an exon. In some embodiments, tertiary and/or quaternary structure affects the tendency of a base to be mutated by a particular mutational process, such as mismatch repair deficiency and others. In some embodiments, the somatic mutations result from mismatch repair deficiency. In some embodiments, the somatic mutations indicate MSI or MSI-H status. In some embodiments, the somatic mutations are MSI or MSI-H mutations.

In some embodiments, primary structure context or sequence context is determined. In some embodiments, secondary structure context is determined. In some embodiments, tertiary structure context is determined. In some embodiments quaternary structure context is determined. Any structural context and any combination of structural context, such as primary structure, secondary structure, tertiary structure, and quaternary structure, can be determined. In some embodiments, primary structure context, secondary structure context, tertiary structure context, quaternary structure context, distance between an exonic base and a splice site, distance between an exonic base and the nearest splice site, location of a somatic mutation within an exon, or any combination thereof, is determined. Any structural context can provide genomic signatures of somatic mutations that result from particular mutational process, such as mismatch repair deficiency and others. In some embodiments, structural context provides genomic signatures for somatic mutations that result from mismatch repair deficiency.

### MSI Score and Classification

An MSI score can be obtained by applying a rule to the results of the steps that that determine the presence of somatic allele lengths in a plurality of tracts of nucleotide repeats in DNA from a sample such as a patient sample, the presence of somatic mutations in the DNA in regions outside of a plurality of tracts of nucleotide repeats, and the genomic signatures of the somatic mutations found outside of a plurality of tracts of nucleotide repeats. In some embodiments, a sample or tumor from which the sample was taken is classified as microsatellite instability-high (MSI-H), microsatellite-instable (MSI), or microsatellite-stable (MSS) based on the MSI score. In some embodiments, the methods provided herein further comprise preparing a report comprising the MSI score and a classification of the sample as microsatellite instability-high (MSI-H), microsatellite-instable (MSI), or microsatellite-stable (MSS).

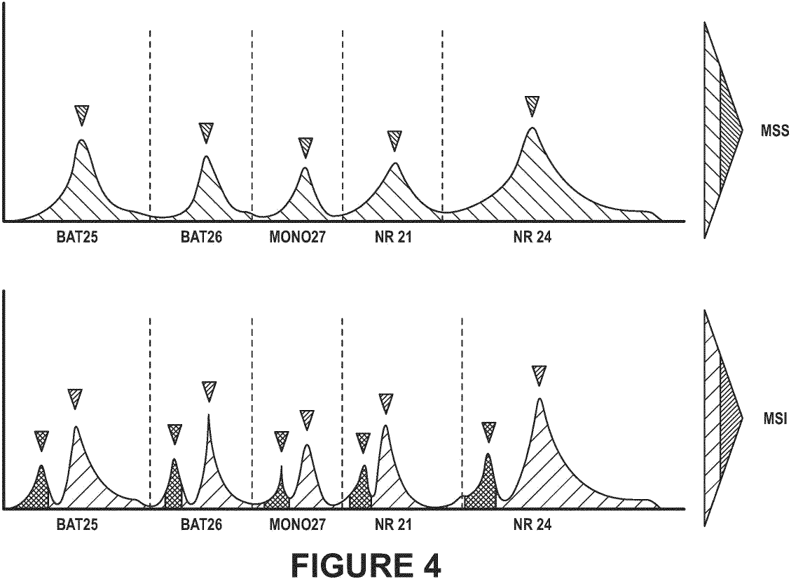

Any microsatellite marker or combination of microsatellite markers can be used to obtain an MSI score, including BAT-25, BAT-26, MONO-27, NR-21, NR-24, BAT-40, TGFβ RII, IGFIIR, hMSH3, BAX and dinucleotide loci such as D2S123, D9S283, D9S1851, D2S123, D17S250, and D18S58, for example. In some embodiments, one or more microsatellite markers are selected from the group consisting of: BAT-25; BAT-26; MONO-27; NR-21; NR-24; and any combination thereof. In some embodiments, one or more microsatellite markers are selected from the group consisting of: BAT-25; BAT-26; D5S346; D2S123; D17S250; and any combination thereof. In some embodiments, one or more microsatellite markers are selected from the group consisting of BAT-25; BAT-26; MONO-27; NR-21; NR-24; D5S346; D2S123; D17S250; BAT40; and any combination thereof. Exemplary microsatellite markers are shown in FIG. 4.

In some embodiments, one or more microsatellite markers are selected from human genome microsatellite markers. Any human microsatellite marker or combinations of human microsatellite markers can be used. Microsatellite markers or combinations of microsatellite markers from species other than humans can also be used, including, for example, microsatellite markers from any mammal such as rodents (including mice, rats, hamsters and guinea pigs), cats, dogs, rabbits, farm animals including cows, horses, goats, sheep, pigs, etc., and primates (including monkeys, chimpanzees, orangutans and gorillas), and others. Unstable tracts of microsatellite markers can be shorter or longer than a reference allele, for example, as described above. Exemplary methods for obtaining an MSI score are described below (Example 3 and Example 4).

### Computing Devices

As one skilled in the art recognizes as necessary or best-suited, performance of the methods provided herein may include one or more computing devices, computing systems, or computers that include one or more of a processor (e.g., a central processing unit (CPU), a graphics processing unit (GPU), etc.), a computer-readable storage device (e.g., main memory, static memory, etc.), or combinations thereof which communicate with each other via a bus.

A processor may include any suitable processor known in the art, such as the processor XEON® E7 by Intel (Santa Clara, Calif.) or the processor sold under the trademark OPTERON™ 6200 by AMD (Sunnyvale, Calif.).

Memory preferably includes at least one tangible, non-transitory medium capable of storing: one or more sets of instructions executable to cause the system to perform functions described herein (e.g., software embodying any methodology or function found herein or computer programs referred to above); data (e.g., images of sources of medication data, personal data, or a database of medications); or both. While the computer-readable storage device can, in an exemplary embodiment, be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, and any other tangible storage media.

Any suitable services can be used for storage such as, for example, Amazon Web Services, memory of the computing system, cloud storage, a server, or other computer-readable storage.

Input/output devices according to the methods provided herein may include one or more of a display unit (e.g., a liquid crystal display (LCD) or a cathode ray tube (CRT) monitor), an alphanumeric input device (e.g., a keyboard), a cursor control device (e.g., a mouse or trackpad), a disk drive unit, a printer, a signal generation device (e.g., a speaker), a touchscreen, a button, an accelerometer, a microphone, a cellular radio frequency antenna, a network interface device, which can be, for example, a network interface card (NIC), Wi-Fi card, or cellular modem, or any combination thereof.

One of skill in the art will recognize that any suitable development environment or programming language may be employed to implement the methods described herein. For example, methods herein can be implemented using Perl, Python, C++, C#, Java, JavaScript, Visual Basic, Ruby on Rails, Groovy and Grails, or any other suitable tool. For a mobile device, it may be preferred to use native xCode or Android Java.

### Samples

In some embodiments, the methods provided herein include sequenced DNA in a sample obtained from a patient. In some embodiments, the patient suffers from a tumor or cancer. In some embodiments, the sample is a tumor sample. Samples from both solid and liquid tumors can be used in the methods described herein. As used herein, the term “tumor” refers to a mass or lump of tissue that is formed by an accumulation of abnormal cells. A tumor can be benign (i.e., not cancer), malignant (i.e., cancer), or premalignant (i.e., precancerous). The terms “tumor” and “neoplasm” can be used interchangeably. Generally, a cancerous tumor is malignant.

As used herein, the term “solid tumor” refers to an abnormal mass of tissue that usually does not contain cysts or liquid areas. Exemplary solid tumors include sarcomas and carcinomas, for example. As used herein, the term “liquid tumors” refers to tumors or cancers present in body fluids such as blood and bone marrow. Exemplary liquid tumors include hematopoietic tumors, such as leukemias and lymphomas, notwithstanding the ability of lymphomas to grow as solid tumors by growing in a lymph node, for example. The term “liquid tumor” can be used interchangeably with the term “blood cancer,” unless context clearly indicates otherwise,

A sample from any cancer can be analyzed by the methods provided herein. Any type of cancer can be analyzed by the methods provided herein. In some embodiments, the cancer is selected from breast cancer, pancreatic cancer, lung cancer, melanoma, skin cancer, hematopoietic cancer, leukemia, lymphoma, colon cancer, rectal cancer, kidney cancer, renal cancer, urinary bladder cancer, oral cavity cancer, pharynx cancer, thyroid cancer, head and neck cancer, brain cancer, bone cancer, muscle cancer, sarcoma, rhabdomyosarcoma, ovarian cancer, cervical cancer, uterine cancer, prostate cancer, and others. Accordingly, the patient of the methods provided herein may suffer from any cancer. In some embodiments, the patient suffers from breast cancer, pancreatic cancer, lung cancer, melanoma, skin cancer, hematopoietic cancer, leukemia, lymphoma, colon cancer, rectal cancer, kidney cancer, renal cancer, urinary bladder cancer, oral cavity cancer, pharynx cancer, thyroid cancer, head and neck cancer, brain cancer, bone cancer, muscle cancer, sarcoma, rhabdomyosarcoma, ovarian cancer, cervical cancer, uterine cancer, prostate cancer, or any other cancer.

Any sample or type of sample can be used in the methods provided herein. In some embodiments, the sample is blood, saliva, plasma, serum, urine, or other biological fluid. Additional exemplary biological fluids include serosal fluid, lymph, cerebrospinal fluid, mucosal secretion, vaginal fluid, ascites fluid, pleural fluid, pericardial fluid, peritoneal fluid, and abdominal fluid. In some embodiments, the sample is a tissue sample. In some embodiments, the sample is a tissue sample from a cancer. In some embodiments, the sample is a cell sample. In some embodiments, the sample is a cell sample from a cancer. In some embodiments, the sample is a cancer sample. A cancer sample can be a sample from a solid tumor or a liquid tumor.

### Patient Selection and Methods of Treatment

In some embodiments, the methods provided herein include selecting or administering a treatment to a patient based on the MSI score. In some embodiments, the patient suffers from cancer. In some embodiments, the MSI score indicates that the sample of the patient is microsatellite instability-high (MSI-H) or microsatellite-instable (MSI) and the treatment comprises immunotherapy. A patient can be selected for treatment with any immunotherapy, as described below. In some embodiments, the MSI score indicates that the sample of the patient is microsatellite instability-high (MSI-H) or microsatellite-instable (MSI) and the treatment comprises an immune checkpoint inhibitor. In some embodiments, the immune checkpoint inhibitor includes an antibody, as described below. In some embodiments, the antibody is selected from the group consisting of: an anti-PD-1 antibody; an anti-IDO antibody; an anti-CTLA-4 antibody; an anti-PD-L1 antibody; and an anti-LAG-3 antibody. In some embodiments, the checkpoint inhibitor is Pembrolizumab (KEYTRUDA®), Nivolumab (OPDIVO®), Atezolizumab (TECENTRIQ®), or Ipilimumab (YERVOY®).

### Immunotherapy

Immunotherapy includes treatment with activation immunotherapies and treatment with suppression immunotherapies. Activation immunotherapies elicit or activate an immune response, while suppression immunotherapies reduce or suppress an immune response. Immunotherapy can include treatment with immune modulators, such as interleukins, cytokines, chemokines, immunomodulatory imide drugs (IMiDs), and others. Any interleukin, cytokine, chemokine, or immunomodulatory imide drug (IMiD) can be used for immunotherapy. Exemplary interleukins for immunotherapy include IL-1, IL-2, IL-3, IL-4, IL-5, IL-6, IL-7, IL-8, IL-10, IL-12, IL-15, IL-18, IL-21, and IL-23. Exemplary cytokines for immunotherapy include interferons, TNF-α, TGF-β, G-CSF, and GM-CSF. Exemplary chemokines for immunotherapy include CCL3, CCL26, and CXCL7. Exemplary IMiDs include thalidomide and its analogues lenalidomide, pomalidomide, and apremilast. Other immunomodulators include cytosine phosphate-guanosine, oligodeoxynucleotides, and glucans, for example.

Cancer immunotherapy generally involves stimulation of the immune system to destroy cancer cells and tumors. Exemplary cancer immunotherapy includes CAR T-cell therapy that introduces chimeric antigen receptors (CARs) to a patient’s T cells to generate CAR-T cells. CAR-T cells are then introduced into the patient’s bloodstream to treat cancer by adoptive cell transfer (ACT). CARs generally include antigen recognition domains that can target antigens expressed on the cell surface of cancer cells and one or more signaling domains. Thus, CAR-T cells can target and destroy cancer cells that express a target antigen. Exemplary CAR-T cell therapies include tisagenlecleucel (KYMRIAH®) and axicabtagene ciloleucel (YESCARTA).

A further cancer immunotherapy includes TCR therapy, another type of ACT. Similar to CAR-T cell therapy, T cells are taken from a patient, reengineered, and introduced to the patient. A further type of ACT includes tumor-infiltrating lymphocyte (TIL) therapy. TILs from a patient are isolated from a patient’s tumor tissue and expanded in vitro, followed by introduction into the patient.

Yet another type of cancer immunotherapy is treatment with monoclonal antibodies. Monoclonal antibodies for use in immunotherapy can be naked, i.e., non-conjugated, or conjugated, i.e., have a chemotherapy drug or radioactive particle attached to them. In addition to monoclonal antibodies, other molecules such as interleukins and cytokines, for example, can be conjugated for targeting cancer cells. As an example, denileukin diftitix (ONTAK) includes IL-2 attached to diphtheria toxin. Further, monoclonal antibodies for cancer immunotherapy can be bispecific, i.e., designed to recognize and bind to two different proteins. Thus, bispecific monoclonal antibodies can recognize more than one antigen on the surface of a cancer cell, for example. As another example, a bispecific antibody can recognize a protein or antigen on a cancer cell and a protein or antigen on an immune cell, thereby promoting the immune cell to attack the cancer cell.

Exemplary monoclonal antibodies for treating cancer include alemtuzumab (CAMPATH), trastuzumab (HERCEPTIN®), ibritumomab tiuxetan (ZEVALIN), brentuximab vedotin (ADCETRIS®), ado-trastuzumab emtansine (KADCYLA®), blinatumomab (BLINCYTO®), bevacizumab (AVASTIN®), and cetuximab (ERBITUX).

Further cancer immunotherapies include cancer vaccines that elicit an immune response against cancer cells. Yet another cancer immunotherapy is “checkpoint inhibitor therapy,” as described further below.

### Checkpoint Inhibitor Therapy

Checkpoint inhibitor therapy is a form of cancer treatment that uses or targets immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. Without being limited by theory, PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells). PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, for example, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-y. PD-L2 expression is more restricted and is expressed mainly by DCs and a few tumor lines.

PD-L1 is expressed in several cancers. Monoclonal antibodies targeting PD-1 can boost the immune system for the treatment of cancer. Many tumor cells express PD-L1, an immunosuppressive PD-1 ligand; inhibition of the interaction between PD-1 and PD-L1 can enhance T-cell responses in vitro and mediate preclinical antitumor activity.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but generally upregulated in conventional T cells after activation, especially in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. Without being limited by theory, CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

Several checkpoint inhibitors can be used to treat cancer. PD-1 inhibitors include Pembrolizumab (KEYTRUDA®) and Nivolumab (OPDIVO®). PD-L1 inhibitors include Atezolizumab (TECENTRIQ®), Avelumab (BAVENCIO®) and Durvalumab (IMFINZI®), for example. CTLA-4 inhibitors include Iplimumab (YERVOY®), for example. Other checkpoint inhibitors include, for example, an anti B7-H3 antibody (MGA271), an anti-KIR antibody (Lirilumab), and an anti-LAG3 antibody (BMS-986016). Any checkpoint inhibitor can be used in the methods described herein. Further, the response to any checkpoint inhibitor can be determined or predicted using the methods described herein. In some embodiments, the checkpoint inhibitor is Pembrolizumab (KEYTRUDA®), Nivolumab (OPDIVO®), Atezolizumab (TECENTRIQ®), Avelumab (BAVENCIO®), Durvalumab (IMFINZI®), or Ipilimumab (YERVOY®).

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20%, or ±10%, or ±5%, or even ±1% from the specified value, as such variations are appropriate for the disclosed methods or to perform the disclosed methods. The term “about” can be used interchangeably with the term “approximately,” unless clearly contradicted by context.

Unless defined otherwise, all technical and scientific terms used herein have the same meaning as is commonly understood by one of skill in the art to which this invention belongs.

As used herein, the term “protein” refers to any polymeric chain of amino acids. The terms “peptide” and “polypeptide” are used interchangeably with the term “protein” and also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA(cfDNA), mRNA, tRNA, rRNA, siRNA, micro RNA (miRNA or miR), hnRNA. Exemplary nucleic analogues include peptide nucleic acid, morpholino- and locked nucleic acid, glycol nucleic acid, and threose nucleic acid.

As used herein, the term “patient” refers to any individual or subject on which the methods disclosed herein are performed. The term “patient” can be used interchangeably with the term “individual” or “subject.” The patient can be a human, although as will be appreciated by those in the art that the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, treatment is administered to a subject at risk of developing a particular disease, or to a subject reporting one or more of the physiological symptoms of a disease, even though a diagnosis of this disease may not have been made. Methods of the present disclosure may be used with any mammal or other animal. In some cases, treatment can result in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “sample” and “biological sample” refer to any sample suitable for the methods provided herein. A sample used in the present methods can be obtained from tissue samples or bodily fluid from a subject, or tissue obtained by a biopsy procedure (e.g., a needle biopsy) or a surgical procedure. In certain embodiments, the biological sample of the present methods is a sample of bodily fluid, e.g., cerebrospinal fluid (CSF), blood, serum, plasma, urine, saliva, tears, and ascites, for example. A sample of bodily fluid can be collected by any suitable method known to a person of skill in the art.

### EXAMPLE 1

This example illustrates mutation signature modeling of MSI-related mutations.

Mutagenic processes tend to have a certain “signature,” or a tendency to mutate specific reference alleles into specific alternate alleles when those reference alleles are surrounded by a specific sequence context. Microsatellite instability (MSI) is caused by mismatch repair deficiency, which has had at least 4 known signatures detailed publicly in a collection of known cancer signatures. A model was created based on position weight matrices (PWMs) that uses these signatures and can indicate the likelihood of a mutation being from an MSI or MSI-H tumor vs. a microsatellite-stable (MSS) tumor. This model can then be used to score mutations in a tumor, and the sum of a tumor’s mutational scores yields a “tumor signature score” that can be used to determine if the tumor itself is MSI or MSS. This score can be used alone or in an ensemble method to perform MSI/MSS classification.

The model of the method is fit to publicly available mutation data (TCGA MC3; Ellrott, K., et al. Scalable Open Science Approach for Mutation Calling of Tumor Exomes Using Multiple Genomic Pipelines. Cell Systems 2018). The model is a set of position weight matrices that takes as input a particular substitution (e.g., C>A, C>G, T>A) and 11 bp context (e.g., CATGCCCAGTC (SEQ ID NO: 1)) centered on the reference allele. The model’s output is a score that is positive when the mutation is more likely to have come from an MSI tumor than an MSS tumor, and is negative when the mutation is more likely to have come from an MSS tumor than an MSI tumor. Software was written to perform the model fitting and scoring.

The model was fit to publicly available exome somatic substitution data, and used the publicly available MOSAIC MSI classification labelling (Hause R., et al. Classification and characterization of microsatellite instability across 18 cancer types. Nat Med 2016) to assign MSI/MSS labels to individual exomes. Mutations from the MSI exomes were used to create a set of 6 position probability matrices (PPMs) (one per possible substitution of a pyrimidine reference base, i.e., C>A, C>G, C>T, T>A, T>C, and T>G); the same was done separately with the mutations from MSS exomes. These two sets of PPMs were combined into one set of 6 PWMs, by dividing entrywise the MSI PPM for a given substitution by the corresponding MSS PPM for that same substitution, and then storing the logarithm of each quotient in the various entries of the PWMs. The PPMs and PWMs used are based on the frequency of overlapping triplets rather than the single characters that traditional PPMs/PWMs use, in order to better account for the effects that adjacent bases can have on the mutation signature. Overlapping triplets can be used as an optional feature to further enhance traditional PWM implementations.

### EXAMPLE 2

This example describes MSI signature scoring of individual mutations and tumors.

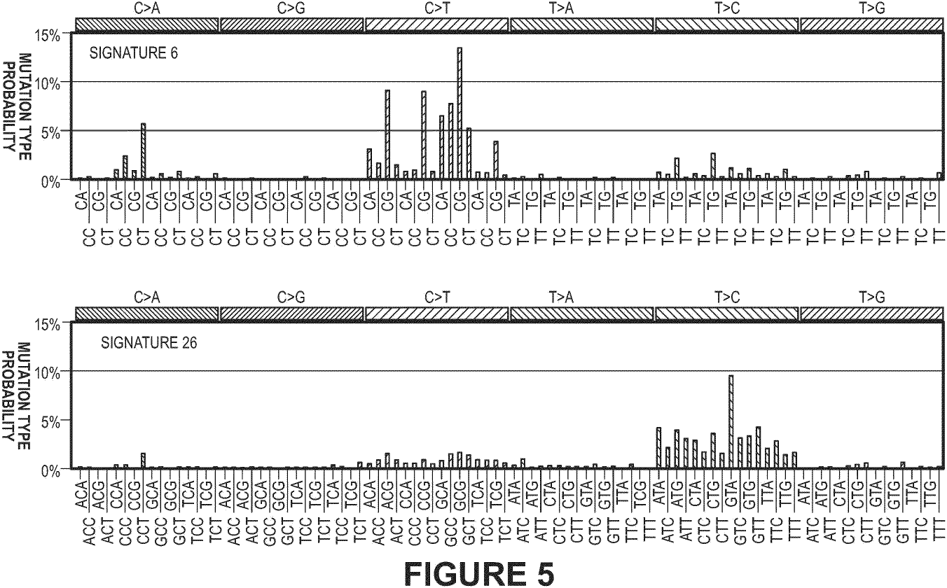

Without being limited by theory, microsatellite instability is caused by mismatch repair deficiency. Mismatch repair deficiency can cause certain somatic mutations to occur in certain contexts (FIG. 5). A model was created that estimates the likelihood that a substitution comes from an MSI or MSI-H tumor or an MSS tumor.

A set of position weight matrices (PWMs) was created that evaluates the log-likelihood that a particular single base substitution (SBS) is from an MSI tumor given the reference allele, alternate allele, and 11 nt context centered on the reference base. This set of matrices was developed from TCGA MC3 data, using the MOSAIC classifications to determine whether a given case was MSI/MSI-H or MSS. The matrix set is used to score all SBSs in a tumor, and the scores are summed from those SBSs to obtain a signature score for the tumor itself. This signature score is then combined with a tract-based score (the percentage of mononucleotide tracts that appear to have a somatic deletion) to get an overall MSI score, which is used to determine the classification for the tumor.

### Structure of a Position Weight Matrix

PWMs were built from a background and a foreground position probability matrix (PPM). The background matrices were developed from substitutions in MSS cases, and the foreground matrices were developed from substitutions in MSI cases. The PWM was created by dividing the probabilities in the foreground matrix by the corresponding probabilities in the background matrix, and calculating the common (base 10) logarithm of the quotient.

Each column of PWMs represents a 3 nt substring of a mutation’s sequence context, as opposed to the 1 nt that would correspond to a traditional PWM’s column. The overlapping 3 nt substrings (3-mers) are used instead of individual bases to better represent the interaction of adjacent nucleotides in a sequence context.

### Sets of Position Weight Matrices

Requiring that the reference base is a pyrimidine (as is done with COSMIC signatures), there are six possible substitutions: C>A, C>G, C>T, T>A, T>C, and T>G. One PWM is maintained for each of these six substitutions. Additionally, the proportion of substitutions for MSI cases that are C>A, C>G, etc. is recorded; the same is done for MSS substitutions. Similar to the calculation of the PWM, the MSI substitution proportions are divided by the MSS substitution proportions and the common logarithm is calculated to arrive at a proportion score. For example, 1.26% of MSI substitutions are C>G, but 12.01% of MSS substitutions are C>G. Therefore, the proportion score for C>G substitutions is log(0.0126/0.1201) = -0.9792, indicating that C>G substitutions are approximately 10 times more likely to be from MSS cases than MSI cases.

### Scoring a Substitution

A substitution is defined by the reference allele, alternate allele, and the 11 nt context centered on the reference base. By convention, it is assumed that the reference base is the pyrimidine base of the base pair, and the substitution is modified accordingly (e.g., a G>T substitution on the forward strand is treated as a C>A substitution with the context extracted from the reverse strand). The reference and alternate alleles determine the proportion score that is used, and the context is scored using a PWM from the set of PWMs that correspond to the particular substitution. For example, a T>G substitution with the context of CAAAGTGAGGA (SEQ ID NO:2) is scored using the T>G PWM, receives a proportion score of -0.1704, and the context has a score of -0.7253. The mutation’s signature score is the sum of the proportion score and context score. In the previous example, the signature score is -0.8957, indicating the mutation is more likely to be from an MSS case (because the score is negative; MSI substitutions are more likely to be positive).

### Scoring a Patient Sample

A tumor’s signature score is determined by summing the mutation signature scores for all somatic mutations found in a tumor sample. Tumors with signature scores above zero are more likely to be MSI, while tumors with signature scores below zero are more likely to be MSS.

### EXAMPLE 3

This example describes combining signature scores with tract-based scores for determination of MSI.

Determination of microsatellite instability using detection of short alleles at mononucleotide tracts is illustrated in FIG. 4. Exemplary mononucleotide tract loci, including BAT25, BAT26, MONO27, NR21, and NR24, are shown. Detection of mismatch repair deficiency using mutation signatures is shown in FIG. 5. Exemplary mutation signatures are shown.

After examining data for 725 tumors, classification rules were established that combined the signature score information with the tract-based scores into an ensemble approach for the detection of microsatellite instability. The two component scores are largely correlated, with all tumors with a tract-based score of less than 10% having a signature score of less than +10, and all cases with a tract-based score of greater than 25% having a signature score of greater than 0. Neither component score perfectly determined what was likely the correct classification (in some cases, PCR orthogonal data was available, and in others analysis of mutational data was used). A boundary line segment between the points of (0.1, 20) and (0.25, -10) was established in a plane with the tract-based score on the x-axis and the signature score on the y-axis. The slope (-200) and y-intercept (40) of the line containing this segment indicate that points where 200x+y >= 40 are classified as MSI, and points where 200x+y < 40 are classified as MSS. There are also two “indeterminate” regions for which there was no data: (a) the region where x<=0.1 and y>=20, and (b) the region where x>=0.25 and y<=-10. Tumors with component scores that fall in an indeterminate region do not have a classification assigned. Reduction of the area of indeterminate regions may change the slope, intercept, and/or endpoints of the boundary line segment.

### EXAMPLE 4

This example describes detection of microsatellite instability using PGDx elio™ tissue complete.

The PGDx elio™ tissue complete pipeline identifies microsatellite instability based on select mononucleotide tracts and genomic context signatures of sequence mutations. A linear classifier determines an overall case status of microsatellite instability-high (MSI-H), microsatellite stable (MSS), or indeterminate by combining the frequency of unstable tracts and signatures of observed mutations.

To determine unstable tracts, a peak finding algorithm was used to determine observed tract lengths for 68 mononucleotide tracts across the region of interest (ROI). Allele lengths for individual tracts were compared to reference lengths to determine unstable tracts that are >2 bp shorter than reference lengths. While lengthening of tracts has been reported, unstable mononucleotide tracts are typically shortened in length, and shifts to longer lengths are uncommon. Systematic lengthening across the mononucleotide tracts of a sample has not been observed. The large number of mononucleotide microsatellite tracts evaluated in PGDx elio™ tissue complete ensured that the lengthening of any single tract, or small frequency of tracts, did not prevent detection of microsatellite instability. Low coverage tracts were excluded from analysis.

A genomic signature score was obtained using a set of position-specific weight matrices. These matrices encode the likelihood of observing a mutation in an MSI-H case versus an MSS case, given the mutation’s alternate allele and surrounding 11 bp genomic context. The matrices were developed using MSI-H and MSS status cases from The Cancer Genome Atlas (TCGA). Candidate single base substitutions were filtered based on germline status, functional consequences, mapping quality, and sequence quality metrics. The remaining sequence variants were assigned the genomic signature score indicating whether that variant is associated with an MSI-H phenotype or not. Likelihood scores from candidate single base substitutions were added to obtain an overall genomic signature score for the sample. The genomic signature score was combined with the tract instability frequency to obtain an overall MSI score. This overall MSI score was determined by adding the proportion of unstable tracts multiplied by 200 to the genomic signature score. An overall MSI score > 40 classifies as MSI-H, otherwise the sample is classified as MSS. Samples that significantly disagree in MSI status between tracts and signature scores are reported as indeterminate.

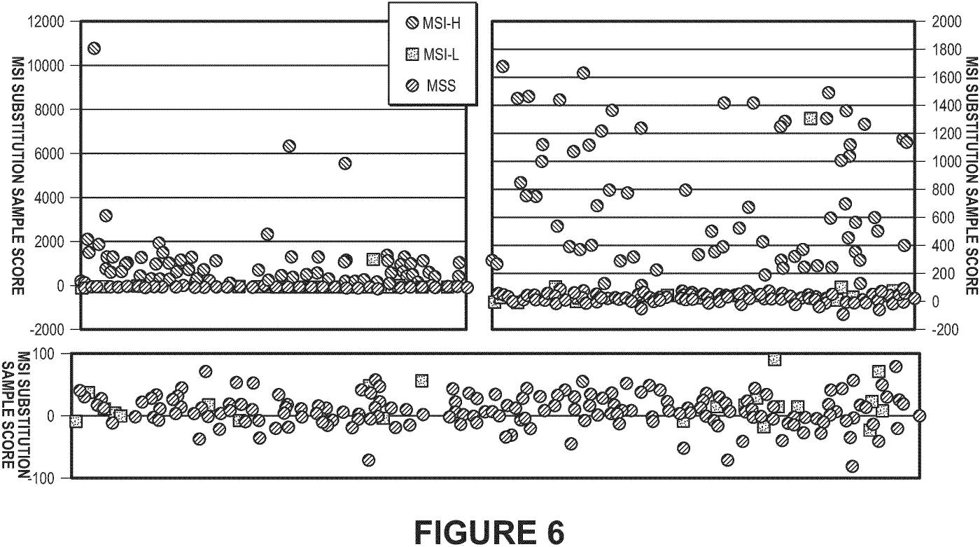

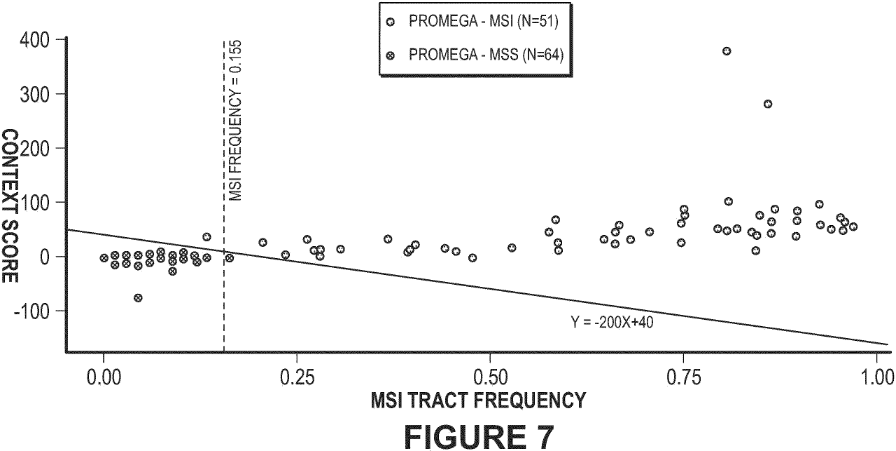

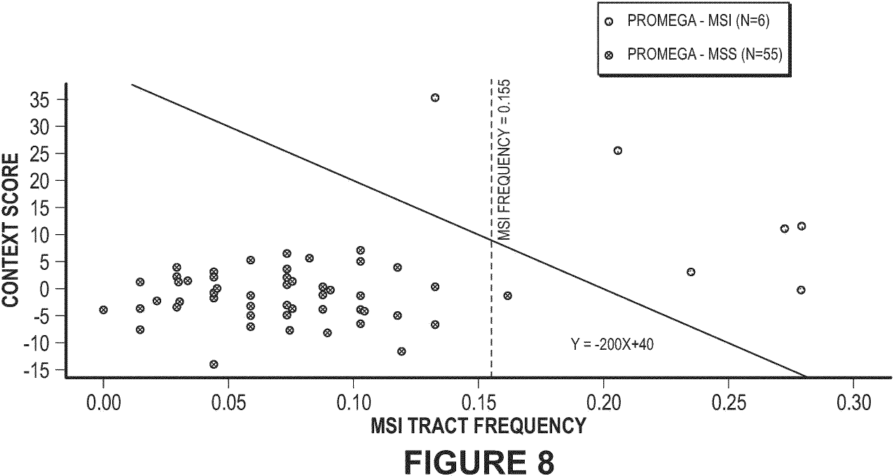

In other studies, it was found that signature scores obtained using exome data appeared concordant with results obtained using PCR for colorectal, gastric, renal, and uterine cancer samples (FIG. 6). Results obtained combining analysis of tracts of nucleotide repeats and genomic signatures of somatic mutations for determination of MSI are shown in FIG. 7 and FIG. 8 that depict samples classified as microsatellite-instable (MSI) or microsatellite-stable (MSS). The data further show that only with both measures, i.e., MSI tract frequency and signature score, does the data become linearly separable.

Taken together, these data show that an ensemble method for the detection of microsatellite instability (MSI) that combines MSI tract frequency and somatic mutation signature scores allows for the classification of samples as MSI/MSI-H or MSS with greater accuracy than methods that rely on a single parameter or score.

Although the invention has been described with reference to the above examples, it will be understood that modifications and variations are encompassed within the spirit and scope of the invention. Accordingly, the invention is limited only by the following claims.

What is claimed is:

1. A method of determining microsatellite instability (MSI) comprising:
(i) determining the presence of somatic allele lengths in a plurality of tracts of nucleotide repeats in sequenced DNA in a sample obtained from a subject;
(ii) determining the presence of somatic mutations in the DNA in regions outside of the plurality of tracts of nucleotide repeats;
(iii) determining a fit of genomic signatures of the somatic mutations found outside of the plurality of tracts of nucleotide repeats to genomic signatures of mismatch repair deficiency;
(iv) applying a rule to the results of the determining steps to obtain an MSI score; and
(v) classifying the sample as microsatellite instability-high (MSI-H) or microsatellite-stable (MSS) based on the MSI score.

2. The method of claim 1, further comprising preparing a report comprising the MSI score and a classification of the sample as microsatellite instability-high (MSI-H) or microsatellite-stable (MSS).

3. The method of claim 1, wherein the sample is a tumor sample.

4. The method of claim 1, wherein the plurality of tracts of nucleotide repeats comprises mononucleotide repeats, dinucleotide repeats, trinucleotide repeats, tetranucleotide repeats, pentanucleotide repeats, hexanucleotide repeats, heptanucleotide repeats, octanucleotide repeats, or combinations thereof.

5. The method of claim 4, wherein the plurality of tracts of nucleotide repeats comprises mononucleotide repeats.

6. The method of claim 1, wherein tracts of nucleotide repeats in MSI-H samples are shorter relative to tracts of nucleotide repeats in reference samples.

7. The method of claim 1, wherein tracts of nucleotide repeats in MSI-H samples are at least two base pairs shorter relative to tracts of nucleotide repeats in reference samples.

8. The method of claim 6, wherein the reference samples are matched normal samples or MSS samples.

9. The method of claim 6, further comprising determining a frequency of shorter tracts of nucleotide repeats.

10. The method of claim 1, wherein the plurality of tracts of nucleotide repeats comprises one or more microsatellite markers.

11. The method of claim 10, wherein the one or more microsatellite markers are selected from human genome microsatellite markers.

12. The method of claim 1, wherein the genomic signatures of the somatic mutations comprise eleven base pairs surrounding the somatic mutations.

13. The method of claim 12, further comprising assigning a genomic signature score to the somatic mutations.

14. The method of claim 13, wherein somatic mutations are classified as associated with an MSI-H sample or an MSS sample based on the genomic signature score.

15. The method of claim 1, further comprising selecting or administering a treatment to the patient based on the MSI score.

16. The method of claim 15, wherein the MSI score indicates that the sample of the patient is microsatellite instability-high (MSI-H) and the treatment comprises an immune checkpoint inhibitor.

17. The method of claim 16, wherein the immune checkpoint inhibitor comprises an antibody.

18. The method of claim 17, wherein the antibody is selected from the group consisting of: an anti-PD-1 antibody; an anti-IDO antibody; an anti-CTLA-4 antibody; an anti-PD-L1 antibody; and an anti-LAG-3 antibody.

19. The method of claim 17, wherein the checkpoint inhibitor is Pembrolizumab Nivolumab, Atezolizumab, or Ipilimumab.

20. The method of claim 1, wherein sequenced DNA in the sample comprises one or more sequenced genomes, one or more sequenced exomes, or regions of one or more sequenced genomes or one or more sequenced exomes.


SEQUENCE LISTING (2 sequences)

SEQ ID NO: 1 - DNA, 11 bp, Artificial Sequence
  Description of Artificial Sequence: Syntheticoligonucleotide
catgcccagtc

SEQ ID NO: 2 - DNA, 11 bp, Artificial Sequence
  Description of Artificial Sequence: Syntheticoligonucleotide
caaagtgagga